(19) United States
(12) Patent Application Publication
(10) Pub. No.: US 2020/0085370 A1
(43) Pub. Date: Mar. 19, 2020

(54) Systems and Methods for Monitoring Breathing and Apnea

(71) Applicant: Montana Technological University, Butte, MT (US)

(72) Inventors: Bryce HILL, Butte, MT (US); Jonathan SCHULZ, Southaven, MS (US)

(73) Assignee: Montana Technological University, Butte, MT (US)

(21) Appl. No.: 16/570,879

(22) Filed: Sep. 13, 2019

Related U.S. Application Data
(60) Provisional application No. 62/730,634, filed on Sep. 13, 2018.

(51) Int. Cl. A61B 5/00; A61B 5/01; A61B 5/08; A61B 5/113; G01S 17/88; G01S 17/02

(52) U.S. Cl. CPC A61B 5/4818; A61B 5/01; A61B 5/0002; A61B 5/0826; A61B 5/1135; G01S 17/88; G01S 17/023; A61B 5/0816

(57) ABSTRACT

Devices and or methods for monitoring respiratory functions of a subject. The system includes a first sensor for measuring the motion of at least on surface of a subject for abdominal, chest, or other area motion to determine volume changes in the area. The second sensor measures the thermal temperature of a target area or device on a surface of the patient, for example near the upper lip of the subject. Specific aspects of the patients breathing health may be analyzed from comparison of respiratory rates calculated from the two sensors. The systems and methods can detect regular breathing, slow breathing (hypopnea), fast breathing (tachypnea), no breathing (apnea), obstructed breathing, or other anomaly. The systems and method may further be configured to trigger an alarm in predetermined situations.

### REFERENCE TO RELATED APPLICATIONS

This application claims the benefit of U.S. Provisional Application No. 62/730,634, filed Sep. 13, 2018, the contents of which are hereby incorporated by reference in their entirety.

### BACKGROUND

Many methods are available to monitor respiratory activity. The monitoring methods are able to measure regular breathing, slow breathing (hypopnea), fast breathing (tachypnea), no breathing (apnea), or obstructed breathing.

Current research into measuring respiratory rate includes remotely measuring the temperature of the upper lip. Though this technology is currently in development, measuring temperature at the lip tracks nasal breathing but does not track oral breathing. The thermal change at the upper lip also may not reflect a significant change due to the thermal properties of human skin and its elevated temperature.

A significant problem with all respiratory monitors is that they fail to determine the difference between respiratory depression, when the drive to breathe causes apnea, and respiratory obstruction, where the patient continues to try to breathe but the upper airway is constricted and the lungs are not ventilated. Monitoring both conditions provides much more accurate detection of breathing abnormalities.

### SUMMARY

The System for Remotely Monitoring Breathing and Apnea described herein uses a combination of at least two sensors to calculate to respiratory rates allowing calculation of the difference between respiratory obstruction and respiratory depression. A first sensor measures the temperature at the upper lip either directly or through a mask or target that changes temperature rapidly. This sensor can be configured to automatically track the face of the subject and point the temperature measurement at the correct location. A second sensor is a LIDAR capable of measuring accurate distances. In this case it would measure the distance between the LIDAR and the thoracic and abdomen of the subject. As the subject breathes the difference over time of the thoracic and abdomen distances can be calculated from an array of points to find the respiratory effort of the subject. This produces an approximate volume change of the thoracic and abdominal volumes.

The present application is directed to System for monitoring subject breathing. Using both the temperatures and the LIDAR measurements the difference between respiratory obstruction and respiratory depression can be analyzed. The analysis compares the respiratory rate of the LIDAR measurements of respiratory rate to the thermal measurements of respiratory rate. If the respiratory rate detected by the thermal sensor and the respiratory rate detected by the LIDAR sensor both detect hypopnea or apnea, then respiratory depression is present. If only the thermal sensor detects hypopnea or apnea, then respiratory obstruction is present.

The methods described herein may include calculating the respiratory rate using a thermal sensor aimed at the face or upper lip of the subject. A rising temperature would indicate an expiratory breath cycle and a dropping temperature would indicate an inspiratory breath cycle. A series falling temperatures that contain one or more temperatures that go below either a calculated or pre-determined threshold indicates the beginning of a breath and a series of temperatures that contain at least one temperature that rises across a threshold temperature indicates the end of a breath.

The method of determining a thermal respiratory rate may include calculating temperature threshold for the determination of the respiratory rate relative to that threshold. This threshold may be calculated using the average ambient temperature and the statistics of the respiratory temperature.

This method of measuring the thermal respiratory rate may include a paint, target, mask or other method to cover the nose and/or mouth for one of two purposes. One purpose is to provide a material that has a high thermal conductivity. Another purpose is to provide a target for an optical camera system to track the optimal target for measuring temperature.

This method may include an optical or LIDAR system to track the face, abdomen, thorax, or more than one of these regions, of the subject. An optical or LIDAR system would use facial tracking software, or track markers placed on the subject that are highly reflective or easy to track for the given system or method.

The method may include calculating respiratory rate using a LIDAR system. The LIDAR would measure the distance between itself and the subject over an array of points on the surface of a patient. The LIDAR would update the distances between the LIDAR and the patient at a regular rate. Using the differences in such measurements of the thoracic and abdominal volumes over time can be used to estimate the overall change in respiratory volume over time and thus the breathing patterns of the patient.

This method may include the comparison of the respiratory rates of the thermal calculation of respiratory rate to the LIDAR calculated respiratory rate. A method for comparison produces warnings and alarms of respiratory anomalies including apnea or other respiratory functions.

### BRIEF DESCRIPTION OF THE DRAWINGS

FIG. 1 shows an example schematic diagram of one embodiment of the system with a LIDAR device and thermal sensor on gimbal devices.

FIG. 2 is an example method for receiving, analyzing, and producing output of breathing anomalies from thermal and LIDAR data.

FIG. 3 is an example method, shown in more detail, of the analysis method for detecting breathing anomalies using LIDAR data.

FIG. 4 is an example method, shown in more detail, of the analysis method for detecting breathing anomalies using temperature data.

### DETAILED DESCRIPTION

The systems and methods herein remotely sense and analyze the respiratory rate of a subject, such as a person, dog, or other breathing creature. The embodiments of the system describe here operate a distance up to two meters in distance but other embodiments of the system may operate at greater distances. The combination of the at least two sensors detect the difference between breathing patterns such as obstructive and depressive apnea in a subject. A first sensor may be used to remotely detect a series of temperatures measured on, for example, the upper lip of the subject. The upper lip may be covered by a mask or other device that allows for visual tracking of the upper lip as well as increased sensitivity to temperature changes from the respiratory flow. A second sensor, using LIDAR technology for example, measures the distance from the sensor to the subject with high precision. This sensor may scan the thoracic and abdominal areas to measure the volume change of those areas which represent the amount of air inhaled and exhaled.

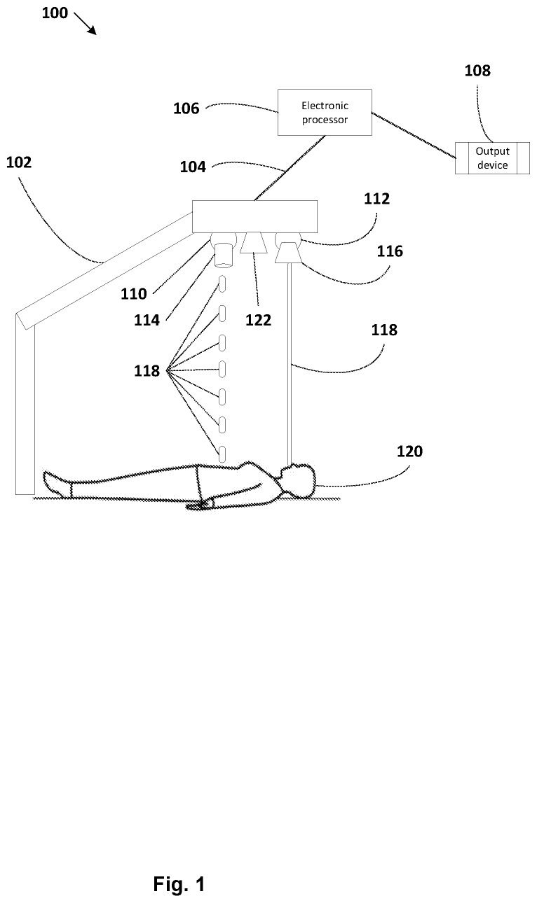

FIG. 1 shows an example embodiment of the system for remotely monitoring breathing and apnea 100. The system for remotely monitoring breathing and apnea 100 includes a support structure 102, an electronic connection 104, an electronic processor 106, and an output device 108. A first gimbal device 110 and a second gimbal device 112 are attached to the support structure 102. A LIDAR device 114 is attached to the first gimbal device 110 and a temperature sensor 116 is attached to the second gimbal device 112 in this example embodiment. The LIDAR device 114 emits or transmits a series of light impulses 118 which reflect or otherwise bounce off a surface of the subject 120 and back to the LIDAR device 114. The temperature sensor 116 uses a sensing technology 118 to sense temperature on a surface of the subject 120. The sensing technology may be a Thermocouple, Resistive Temperature Device (RTD), Thermistor, Integrated Silicon Based Sensors, Infrared (Pyrometers), Thermal Pile technology, or another type of technology to measure temperature of one or more location on the surface of the subject 120.

The LIDAR device 114 is directed by the first gimbal device 110 to be pointed at a surface on the subject 120 to measure movement of the surface of the subject 120. Measurement data capturing movements of the surface of the subject is sent to the electronic processor 106. Similarly, the temperature sensor 116 is directed by the second gimbal device 112 to be pointed at a surface on the subject 120 to measure thermal respiratory flow data, where such respiratory flow data is transmitted to the electronic processor 106. The methods of transmitting the data may include, but are not limited to, metallic wires such as twisted pair or coaxial cable, fiber optic cable, electromagnetic channels such as IEEE 802.11, Bluetooth, cellular channels, or any other wireless communication method. The electronic processor 106 analyzes measurement data and thermal respiratory flow data received and if anomalies are detected, sends a signal to output device 108 to alert a user to such anomaly. The system for remotely monitoring breathing and apnea 100 may, in some embodiments, include a camera 122 which sends subject positioning data to the electronic processor 106, wherein the electronic processor 106 sends signals to the first gimbal device 110, the second gimbal devices 112, or both, to reposition the LIDAR device 114, the temperature sensor 116, or both to obtain better quality data from the LIDAR device 114, the temperature sensor 116, or both.

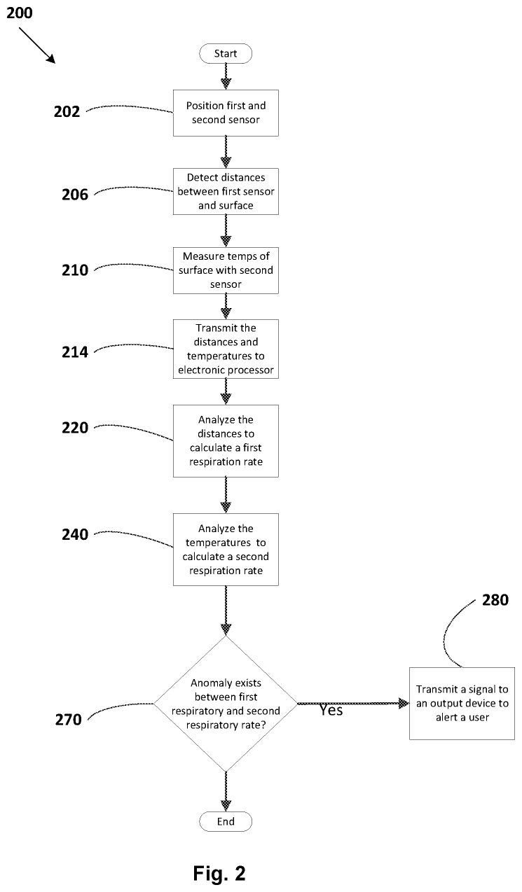

FIG. 2 shows an example method remotely monitoring breathing 200 implemented by the example embodiment of the system for remotely monitoring breathing and apnea 100 shown in FIG. 2. The method remotely monitoring breathing 200 starts by positioning a first sensor and a second sensor to acquire data from a subject (at block 202). The first sensor detects a series of distances between the sensor and a surface of a subject over a period of time (at block 206). The second sensor, similarly, detects a series of temperatures from the surface of a subject over a period of time (at block 210). The series of distances and series of temperatures are sent to the electronic processor (at block 220). The electronic processor analyzes the series of distances to determine a first respiratory rate (at block 220) and analyzes the series of temperatures to determine a second respiratory rate (at block 240). The first respiratory rate and second respiratory rate are compared (at block 270) and if an anomaly exists between the first respiratory rate and second respiratory rate, the electronic processor transmits a signal to an output device to alert a user (at block 280). In some embodiments comparison of first respiratory rate to the second respiratory rate may be based on a tolerable difference, a comparison of patterns in the series of data, or another method of comparison to determine if an anomaly exists. It should be understood that the comparison criteria may vary based on characteristics of the subject, such age, weight, physical condition, environment, or other factors, or may combine multiple factors in such comparison.

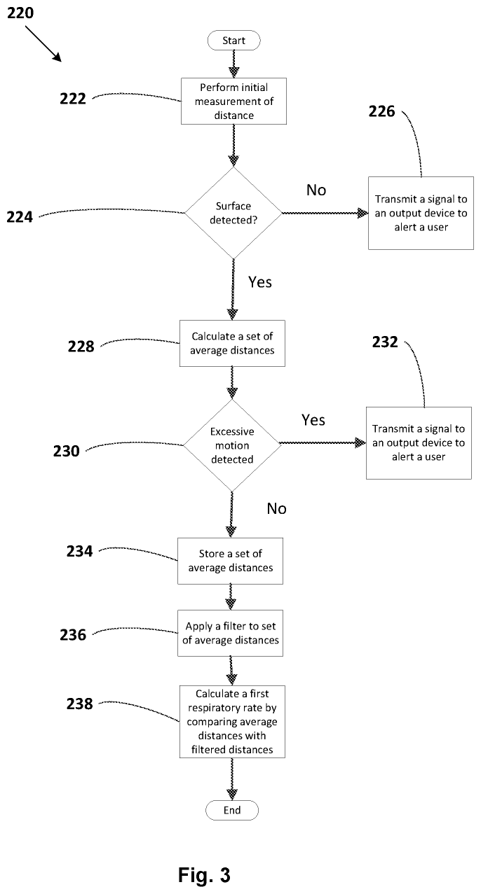

FIG. 3 shows in more detail an example of analyzing the distances to calculate a first respiration rate (block 220) implemented by the example embodiment of the system for remotely monitoring breathing and apnea 100 using the example method shown in FIG. 2. The more detailed example method for analyzing the distances to calculate a first respiration rate (block 220) includes performing an initial measurement of the distance between the LIDAR sensor device 114 and the surface of the subject (at block 222) to establish a baseline measurement for the series of measurements. If a distance has not been measured, meaning a surface is not detected (at block 224) then a signal is sent to the output device 118 to alert a user. If a distance has been measured, meaning a surface has been detected, a set of average distances is calculated using the series of distances (at block 228). If the average set of distances show large variability, significant changes between average distances, or exceed other preset or calculated thresholds, excessive motion is detected (at block 230) and a signal is sent to the output device 118 (at block 232). If excessive motion is not detected (at block 230), the set of average distances is stored for further analysis (at block 234). A filter may be applied to the average distances to determine to extract or convert the average distances to filtered or converted values (at block 236) that can be used to calculate the distance respiratory rate based on the filtered or converted distances compared with average distances (at block 238).

It should be recognized that average distances can be calculated over a series of distance values, a subseries, or multiple subseries. The subseries may overlap or be disjoint and the method described here includes all such series and subseries of distance values. One skilled in the art will also recognize comparison between filtered or converted distance or average distance values and average distance values can be done over the same series, same subseries, or between different distinct or overlapping subseries. The method described here is not limited to any selection of series of filtered or converted average distances or raw distance values detected.

The example method described here to measure distance respiratory rate is based on LIDAR technology. Many types of LIDAR devices are available and provide a wide variety of data and neither the methods or the example system is limited to any particular LIDAR device or technology. LIDAR can retrieve distance and intensity information using time-of-flight (TOF) of a laser reflecting back to the LIDAR, for example. Using this type of LIDAR, the data is scanned both in angle and in azimuth to create an array of distances and intensities. More simple LIDAR systems may only return distance measurements from a single azimuth angle rather than across a range. The LIDAR technology generally can be aimed at the subject using either the data returned from the LIDAR or using a secondary method such as optical detection that may include, but is not limited to a camera, such as camera 122, using image processing techniques to identify the body position of the subject or markers placed on the subject to focus or aim the LIDAR device 114. LIDAR devices may scan the surface of a subject successively estimating, for example, the thoracic and abdominal volumes and tracks the volume changes over time through the distance measurements. Two different volumes measured over time, where the time may be overlapping or disjoint, can be analyzed together or independently. In one embodiment, for example, two volume estimations are combined to estimate total volume exchange during the period of time one or more breaths are taken by the subject. In still other embodiments the two volumes are measured independently to determine if paradoxical breathing is present.

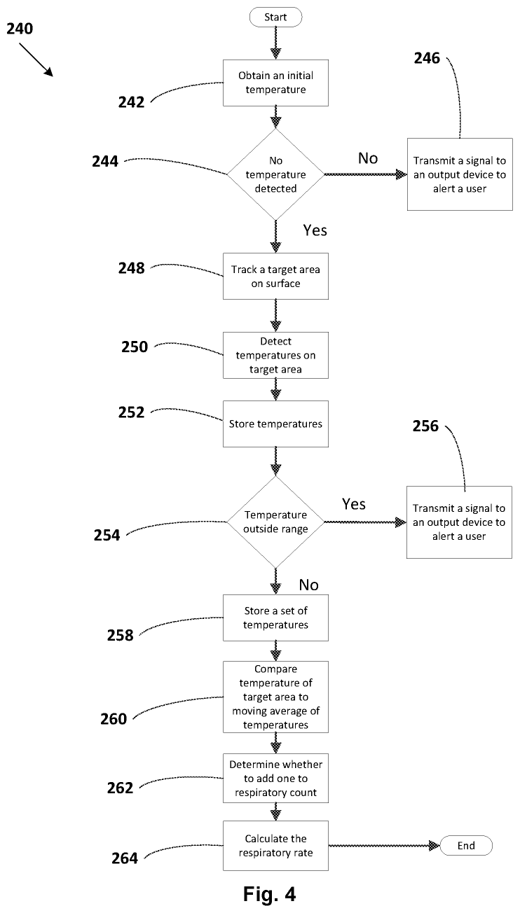

FIG. 4 shows in more detail an example of analyzing the temperatures to calculate a second respiration rate (block 240) implemented by the example embodiment of the system for remotely monitoring breathing and apnea 100 using the example method shown in FIG. 2. The more detailed example method for analyzing the temperatures to calculate a second respiration rate (block 240) includes obtaining an initial measurement of the temperature on the surface of the subject 120 with a temperature sensor device 116 (at block 242). If a temperature has not been detected (at block 244) a signal is sent to the output device 118 to alert the user (at block 246). If a temperature is detected the temperature sensor 116 tracks a target area on the surface where the temperature fluctuates based on respiration of the subject (at block 248). Using data from temperature sensor device 116, detect the temperatures on the surface of the target area (at block 250) and store the detected temperatures (at block 252). Compare the detected temperatures to a preset or calculated temperature range (at block 254) and if the detected temperatures are outside the temperature range transmit a signal to output device 108 (at block 256), otherwise store a set of temperatures (at block 258). The stored set of temperatures are compared to a moving average of temperatures (at block 260), where such moving average can be calculated in a number of ways, including increasing or decreasing the number of temperatures included in the calculation, excluding temperatures outside a preset range, or using other methods that weight temperatures based on their position in a time series of detected temperatures. Through comparison, determine whether to add to the respiratory count for a selected time period (at block 262). Using the respiratory count, a respiratory rate can be calculated for the subject (at block 264).

The thermal respiratory rate analyzer utilizes a temperature sensor 116 that can detect the temperature of a surface at a distance. Various types of temperature sensors may be used for measuring the temperature around a surface, including for example the area of the upper lip of the subject. An optical method of detecting the face and/or a target placed on the face may be employed to aim the temperature sensor 116 at the target area, using a camera for instance as described previously. The optical method may include but is not limited to cameras or a second LIDAR device to detect the correct target. Image processing may be employed to identify the correct target area. The temperature sensor 116 will then measure the variation in measured temperature over time on the identified surface without the need to contact the identified surface physically.

One skilled in the art will recognize the system for remotely monitoring breathing and apnea 100 may include multiple cameras positioned in different locations, or an integrated camera system communicating through an electronic processor to the electronic processor 106, and that utilizing image processing to determine the orientation and position of the subject can be accomplished in a variety of way. For example, image processing could be accomplished through the use of markers placed on the subject, or through the use of body recognition technology.

The system may include a LIDAR system utilizing 3D processing of the data to determine the orientation and position of the subject, and could be done in real time so that the data stream was more accurate. Such a system may utilize markers placed on the subject, or utilize more than one LIDAR system, or similar technology, in order to gather data from the subject's surface area. The system described here utilizes such data in conjunction with thermal data, and either data source may utilize alternative technology to provide such data than that shown or described here.

The system gathering thermal data may include a thermal target or mask placed on the face of the subject. This target serves two purposes: create a color coded or identifiable target for the imaging system or LIDAR to track; and act as a thermal conductor from air passing from the mouth and/or nose of the subject. The thermal sensor then gathers temperature data from this point. One skilled in the art recognizes that data may be gathered in a number of ways and that the system described here uses such data to determine breathing abnormalities.

The system may include targets other than the one on the upper lip or face of the subject. These targets can be placed on the subject or near the subject as targets for positioning of the remote sensor array and that the system described here will utilize such data regardless of the system used to gather the data.

The system for remotely monitoring breathing and apnea 100 calculates respiratory rate from two independently acquired data streams, one calculating volume changes, for example using LIDAR, and one calculating temperature change, for example using a thermal sensor. The volume data will use multiple points to find the approximate volume change of the abdomen, chest or both. A moving average of this signal may be calculated to find a respiratory rate threshold. The respiratory rate can be calculated as the approximated volume crosses the moving average to create a crossing from inhale to exhale. Increases in volume indicate inspiratory breaths, and decreases in volume indicate expiratory breaths. A minimum volume change threshold may be used to determine if the breath has taken place. The number of breaths is can then be counted over a pre-determined time period by dividing the number of breaths in that period, by the time from the first breath in the period to the last breath in the window. Other methods may also be used to calculate the respiratory rate from the given volume data including but not limited to Fourier Transform, Laplace Transform, phase angle estimation, or other signal processing methods.

The respiratory rate may be calculated from the thermal sensor by collecting the temperature at the thermal target and comparing the waveform to a predetermined temperature or a temperature calculated from the subject over time as a baseline of normal respiration. For example, the threshold may be set to, for example, 23.3° C. and left unchanged or altered through feedback of the system. The respiratory rate may be calculated as the sensed temperature crosses a moving average to create a crossing. Decreases in temperature indicate inspiratory breaths, and increases in temperature indicate expiratory breaths. A minimum temperature change threshold will be used to determine if the breath has taken place. The number of breaths is then counted over a pre-determined time period by dividing the number of breaths in that period, by the time from the first breath in the period to the last breath in the period. Other methods may also be used to calculate the respiratory rate from the given data including but not limited to Fourier Transform, Laplace Transform, phase angle estimation, or other signal processing methods.

Comparing the respiratory rate of the two methods determines the correlation or other relationship of the respiratory rate calculated by the two methods. At least four possible outcomes exist when comparing the calculated respiratory rates. The first outcome is that the two respiratory rates agree within a margin of error. Alternatively, one measured respiratory rate may be above a rate threshold indicating the subject is breathing adequately. In the second case the two respiratory rates agree as in the first case, but the respiratory rate is lower than an allowable threshold which indicate slowing respiratory rate. This case indicates respiratory depression and an alarm indicating respiratory depression may be triggered. In the third case the volume respiratory rate is higher in value than the thermal respiratory rate. This indicates that the subject is experiencing respiratory obstruction. In this case the thermal respiratory rate is trusted and an alarm indicating respiratory obstruction is triggered. The fourth case is if the thermal respiratory rate is higher in value than the volume respiratory rate calculated. This may indicate shallow breathing, or that the subject is covered too heavily for the LIDAR to measure significant motion, or some other obstruction or condition exists. In this case an alarm indicating a system error may be sent to the output device 108.

Excessive motion of the subject may also be detected by either or both methods. When the changes in the respiratory volumes indicate that significant motion is or has occurred, a warning signal may be triggered to indicate the subject is in distress or another condition requiring attention. In this case the respiratory rates may not be trusted due to the motion of the subject.

Using the volume system, paradoxical breathing may also be detected. Paradoxical breathing occurs when the chest and the abdomen volumes counteract each other. Such episodes can occur when the airway is obstructed. One method of calculating paradoxical breathing is by measuring the phase difference between the volume change in the abdomen and the volume change in the chest, which can be detected by the volume system, or by multiple volume systems. The phase difference between two volume calculations, from one or two volume systems, may indicate paradoxical breathing. Paradoxical breathing may be determined by comparing the volume change of the abdomen, the volume change of the chest, and the combined volume change. The ratio of the combined volume change to each of the abdomen and chest volume changes can indicate the severity of the paradoxical breathing which may be identified as respiratory obstruction.

The volume system can also be used to determine other medical issues other than apnea, and apnea is used herein for illustrative purposes of one of many breathing anomalies that can be detected by the system described here. For example, the electronic processor 106 may utilize signal processing methods against a known database of volume values captured from the volume system or provided from another source to determine if a subject is having a seizure, experiencing convulsions, sleeping restlessly, or other respiratory issues exist based on volume or motion data.

One skilled in the art will recognize the system for remotely monitoring breathing and apnea 100 utilizes volume and temperature data captured from sensors over a time period to detect many conditions of a subject and that the example system embodiments shown here are illustrative and not exhaustive. The system as described may be used in a variety of ways, on a variety of subjects, to detect breathing anomalies.

What is claimed is:

1. A system for monitoring breathing and apnea, the system comprising:
a first sensor that emits a plurality of pulses of laser light directed toward at least one surface and detects a reflection of the plurality of pulses of laser light;
a second sensor that measures a plurality of temperatures on the at least one surface;
a support structure to which at least one gimbal device is attached, wherein the first sensor and the second sensor are attached to the at least one gimbal device;
at least one electronic processor, wherein the processor is communicatively coupled to the first sensor and the second sensor; and
an output device communicatively coupled to the at least one electronic processor.

2. The system of claim 1 wherein the first sensor comprises a Light Detection And Ranging (LiDAR) device.

3. The system of claim 1 wherein the second sensor comprises a narrow beam remote sensor.

4. The system of claim 1 wherein the at least one surface comprises one or more surface selected from: a subject's upper lip, a mask, a subject's thoracic region, and a subject's abdominal region.

5. The system of claim 1 wherein the electronic processor is coupled to the support structure.

6. The system of claim 1 wherein the electronic processor is communicatively coupled to one or both of the first sensor and the second sensor via wireless communication.

7. The system of claim 1 wherein the gimbal device is, at least in part, controlled by movement commands transmitted to the gimbal device from the at least one electronic processor.

8. The system of claim 1, further comprising:
a camera coupled to the support structure; and
an electronic coupling between the camera and the at least one electronic processor;
wherein the at least one electronic processor sends signals to the gimbal device to control the gimbal device.

9. A method for remotely monitoring breathing and apnea, the method comprising:
positioning a first sensor coupled to at least one gimbal device such that the first sensor is directed toward at least one surface;
positioning a second sensor coupled to the at least one gimbal device such that the second sensor is directed to the at least one surface;
emitting pulses of laser light from the first sensor;
receiving at least one reflection of the pulses of laser light;
detecting, using the at least one reflection of the pulses of laser light, a plurality of distances between the first sensor and the at least one surface;
measuring, using the second sensor, a plurality of temperatures on the at least one surface;
transmitting, to at least one electronic processor, the plurality of distances between the first sensor and the at least one surface;
transmitting, to the at least one electronic processor, the plurality of temperatures detected on the at least one surface;
detecting, at least in part by using the electronic processor to analyze the plurality of distances between the first sensor and the at least one surface and the plurality of temperatures detected on the at least one surface, an anomaly on the at least one surface; and
transmitting, from the electronic processor to an output device, a signal to alert a user that an anomaly exists on the at least one surface.

10. The method of claim 9 wherein the first sensor and the second sensor are positioned by the gimbal device which is controlled by the at least one electronic processor, wherein such positioning is determined at least in part by the at least one electronic processor analyzing images of the surface.

11. The method of claim 9 wherein the second sensor uses a plurality of signals from the surface to capture a thermal data stream to calculate a respiratory rate.

12. The method of claim 9 wherein detecting an anomaly on the at least one surface further comprises:
calculating a first respiratory rate, at least in part using the plurality of distances between the first sensor and the at least one surface;
calculating a second respiratory rate, at least in part using the plurality of temperatures on the at least one surface;
analyzing motion at least in part by comparing the first calculated respiratory rate to the second calculated respiratory rate; and
comparing the respiratory rate to at least one previous respiratory rate of a subject to determine if the subject is breathing normally, is in depressive apnea, or is in obstructive apnea.

13. The method of claim 12 wherein the at least one previous respiratory rate is chosen from a group consisting of the subject's previous respiratory rate, another subject's respiratory rate, and the normal respiratory rate of a people similar to the subject.

14. The method of claim 9, wherein the first sensor comprises a Light Detection And Ranging (LiDAR) device.

15. The method of claim 9 wherein the second sensor comprises a narrow beam remote sensor.

16. The method of claim 1 wherein the at least one surface comprises one or more surface selected from: a subject's upper lip, a mask, a subject's thoracic region, and a subject's abdominal region.

17. The method of claim 9, wherein the electronic processor is communicatively coupled to one or both of the first sensor and the second sensor via wireless communication.

18. The method of claim 9 wherein the gimbal device is, at least in part, controlled by movement commands transmitted to the gimbal device from the at least one electronic processor.
